(19) United States
(12) Patent Application Publication
(10) Pub. No.: US 2003/0077816 A1
(43) Pub. Date: Apr. 24, 2003

(54) Bioreactor and method for using

(76) Inventors: Richard Kronenthal, Rutherford, NJ (US); Nitya G. Ray, East Hanover, NJ (US); Kimberly Tahan, Kinnelon, NJ (US); John Wilson, New Brighton, MN (US)

Correspondence Address: Ralph T. Lilore, Attorney-at-Law, 371 Franklin Avenue, Third Floor, P.O. Box 570, Nutley, NJ, 07110, US

(21) Appl. No.: 10/032,925

(22) Filed: Dec. 26, 2001

Related U.S. Application Data
(63) Continuation-in-part of application No. 09/749,226, filed on Dec. 27, 2000, now abandoned.

(51) Int. Cl. C12M 3/04

(52) U.S. Cl. 435/297.2; 435/299.1; 435/177; 435/402

(57) ABSTRACT

A bioreactor is described comprising: at least one cartridge having disposed therein at least one collagen substrate comprised of a collagen sponge layer and a nonporous to cells, semipermeable collagen layer; a substrate support that retains the collagen substrate within the cartridge; a first compartment between the collagen sponge layer and the inner surface of the first side of the cartridge casing and a second compartment between the nonporous to cells, semipermeable collagen layer and the inner surface of the second side of the cartridge casing; inlet and outlet means for transferring a first medium and a first cell type to the first compartment and inlet and outlet means for transferring a second medium and a second cell type to the second compartment. Also described is a method for using the bioreactor to grow a Composite Living Construct (CLC) comprised of a first layer comprising a cultured first cell type and a second layer comprising a cultured second cell type; seeding and culturing the first cell type on and within the collagen sponge layer; and seeding and culturing the second cell type on the nonporous to cells, semipermeable collagen layer. Another embodiment of this invention includes methods for cutting the CLC into sections or units in preparation for product packaging of appropriate sizes. Yet another embodiment of this invention includes equilibration of the CLC with cryoprotectant solutions within the cartridge, further cutting the CLC into sections or units in preparation for cryopreservation. Yet another embodiment of this invention includes equilibration of the CLC with cryoprotectant solutions and cryopreservation within the cartridge, with the cartridge providing product packaging. Yet another embodiment of this invention includes methods for cutting the CLC into sections or units in preparation for product packaging, followed by equilibration with cryoprotectant solutions and cryopreservation within the package.

### RELATED APPLICATIONS

This application is a continuation-in-part of co-pending U.S. Ser. No. 09/749,226 for Bioreactor and Method of Using filed Dec. 27, 2000.

### TECHNICAL FIELD

The present invention relates to bioreactors and methods of using such bioreactors for growing a Composite Living Construct (CLC) comprised of at least a first layer comprising a cultured first cell type such as fibroblasts and at least a second layer comprising a cultured second cell type such as keratinocytes.

### BACKGROUND

Composite Living Constructs (CLCs), belonging to a class of tissue replacements also known as biologically active wound dressings, are distinguished by comprising both a dermal layer comprised of fibroblasts and an epidermal layer comprised of keratinocytes. This invention describes a bioreactor and a process for using such a bioreactor to grow CLCs that are comprised of separate cultured layers of a first cell type such as fibroblasts on and within a collagen sponge layer and a second cell type such as keratinocytes on a nonporous to cells, semipermeable collagen layer, the nonporous collagen layer being semipermeable in that it is impermeable to biological cells and permeable to gases and soluble molecular components. Such CLCs are described in U.S. Pat. Nos. 5,282,859, Re 35,399 and 6,039,859, to Eisenberg, which are incorporated herein by reference. Such CLCs are used, for example, for the treatment of chronic and acute skin wounds in humans or animals. Various biologically active wound dressings may be employed to cover and aid the regeneration of tissue in wound areas such as granulating wounds, deep abrasions, excisions, burns, ischemic skin ulcers and dystrophic epidermolysis bullosa wounds.

Conventional processes of tissue culture are static systems that depend on manual handling, are subject to error and do not permit large scale, automated, reproducible and economical production of tissues. The conventional processes of seeding, culturing, media delivery and waste removal are complex and inefficient.

Optimum designs for and methods for use of a bioreactor are dictated by the cell substrate and cell types that are to be grown therein. Growth conditions and media formulations, including their gaseous components, should be developed and tested, particularly within the confines of the bioreactor. Designs and methods depend on such factors as cell type and substrate characteristics including: configuration, mass, density, permeability, and thickness. Optimum designs and methods also need to accommodate the presence of different cells and their interfaces and interactions within the three dimensional substrate.

### SUMMARY OF THE INVENTION

It is an object of this invention to provide a bioreactor comprising a collagen substrate that divides the bioreactor into first and second compartments and further comprising means for transferring a first medium and a first cell type to the first compartment and means for transferring a second medium and a second cell type to the second compartment.

Another object of this invention is to provide a method of using the bioreactor to grow a Composite Living Construct (CLC) comprised of a first layer comprising a cultured first cell type such as fibroblasts and a second layer comprising a cultured second cell type such as keratinocytes.

Another embodiment of this invention includes methods for cutting the CLC into sections or units in preparation for product packaging of appropriate sizes. Yet another embodiment of this invention includes equilibration of the CLC with cryoprotectant solutions within the cartridge, further cutting the CLC into sections or units in preparation for cryopreservation. Yet another embodiment of this invention includes methods for cutting the CLC into sections or units in preparation for product packaging, followed by equilibration with cryoprotectant solutions and cryopreservation within the package.

One preferred embodiment of this invention provides a bioreactor comprising: at least one cartridge having disposed therein a collagen substrate comprised of a collagen sponge layer and a nonporous to cells, semipermeable collagen layer, each layer having an inner surface in contact with an inner surface of the other layer and an outer surface; a substrate support that retains the collagen substrate within the cartridge; a first compartment defined between the outer surface of the collagen sponge layer and the inner surface of one side of the cartridge casing and a second compartment defined between the outer surface of the nonporous to cells, semipermeable collagen layer and the inner surface of a second side of the cartridge casing; and a first compartment inlet means and outlet means for transferring a first medium and a first cell type to the first compartment and a second compartment inlet means and outlet means for transferring a second medium and a second cell type to the second compartment, the inlet and outlet means being situated so that the media are transferred essentially parallel to the outer surfaces of the layers of the collagen substrate.

The nonporous to cells, semipermeable collagen layer is permeable to gases and soluble molecular components and impermeable to biological cells such as fibroblasts and keratinocytes. The cartridge and the collagen substrate therein are sterilizable. A portion of the surface of the cartridge casing may optionally be comprised of a gas permeable, liquid impermeable membrane to permit aseptic transfer of gases such as air, oxygen and carbon dioxide into and out of the bioreactor.

The substrate supports are preferably grids adjacent the outer surfaces of the collagen sponge layer and nonporous to cells, semipermeable collagen layers that comprise the CLC, the grids being comprised of solid portions and openings therebetween. The openings of the grids preferably are configured to facilitate dividing the CLC into sections or units, the sections or units preferably being of sizes and configurations that are convenient for use such as for wound dressings.

The cartridge may further comprise a frame around the periphery of the substrate support grids and the collagen substrate therebetween to secure the support and substrate within the cartridge. The cartridge may further comprise additional components including, but not limited to, viewing windows, gas transfer membranes, frames, support structures as well as fastening means to secure the components of the cartridge, to: facilitate viewing of the bioreactor contents during use; maintain its structural robustness; facilitate assembly and disassembly of its components; allow gas exchange; and maintain aseptic conditions during its use.

In addition to the inlet and outlet means for the two compartments of the cartridge, the bioreactor may further comprise means for evenly distributing media across the major outer surfaces of the collagen substrate, such as multiple fluid inlet and outlet ports, ribs, channels and wells. The media are such as cell growth media, cell conditioned media, maintenance media, washing media, rinsing media, cell growth factors, enzymes, extracellular matrix components, gases and cryoprotectant solutions.

A multiple cartridge bioreactor system may have the following fluid pathway alternatives: for an independent cartridge arrangement, the fluid flow path to the first compartment of each cartridge is separate from that of the first compartment of the other cartridges and the fluid flow path to the second compartment of each cartridge is separate from that of the second compartment of the other cartridges; for a series cartridge arrangement, the fluid flow path to the first compartment of each cartridge is connected in series and the fluid flow path to the second compartment of each cartridge is connected in series; for a parallel cartridge arrangement, the fluid flow path to the first compartment of each cartridge is connected in parallel and the fluid flow path to the second compartment of each cartridge is connected in parallel; for a combination parallel and series arrangement, groups of cartridges are connected in a parallel arrangement as described above, while the fluid flow path from the first compartment of each of these grouped cartridges is connected in series with the fluid flow path to the respective first compartment of the cartridges in the remaining parallel groups and the fluid flow path from the second compartment of each of these grouped cartridges is connected in series with the fluid flow path to the respective second compartment of each of the cartridges in the remaining parallel groups. The fluid pathways of the first and second compartments of each cartridge optionally may be connected with each other to form a unitary fluid pathway. Optionally, the multiple bioreactor system may have any of the above mentioned fluid flow path arrangements with the exception that the fluid pathway connects only the first compartments of each cartridge, or the fluid pathway connects only the second compartments of each cartridge.

Another preferred embodiment of this invention provides a method of making a CLC comprised of at least a first layer comprising a cultured first cell type and at least a second layer comprising a cultured second cell type, the method comprising the steps of:

1) providing a bioreactor comprising: at least one cartridge having disposed therein a collagen substrate comprised of a collagen sponge layer and a nonporous to cells, semipermeable collagen layer, each layer having an inner surface with an inner surface of the other layer and an outer surface; a substrate support that retains the collagen substrate within the cartridge; a first compartment defined between the outer surface of the collagen sponge layer and the inner surface of the first side of the cartridge casing and a second compartment defined between the outer surface of the nonporous to cells, semipermeable collagen layer and the inner surface of the second side of the cartridge casing; and a first compartment inlet means and outlet means for transferring a first medium and a first cell type to the first compartment and a second compartment inlet means and outlet means for transferring a second medium and a second cell type to the second compartment, the inlet and outlet means being situated so that the media are transferred essentially parallel to the outer surfaces of the layers of the collagen substrate;

2) transferring a first medium and a first cell type to the first compartment;

3) effecting a seeding and culturing of the first cell type on the collagen sponge layer;

4) transferring a second medium and a second cell type to the second compartment; and

5) effecting a seeding and culturing of the second cell type on the nonporous to cells, semipermeable collagen layer.

It is preferred that the steps of transferring the first medium and the first cell type to the first compartment and transferring the second medium and the second cell type to the second compartment each be performed with the major surface of the collagen substrate being between about 45° and 90° from the horizontal, the inlet and outlet means being situated to minimize entrapment of gases within the cartridge. It is further preferred that the steps of effecting a seeding and culturing of the first cell type on the collagen sponge layer and effecting a seeding and culturing of the second cell type on the nonporous to cells, semipermeable collagen layer be each done with the major surface of the collagen substrate being between about 45° and 0° from the horizontal to provide good and even attachment of the cell types to the respective layers of the collagen substrate.

Yet another preferred embodiment of this invention includes further processing of the CLC comprising the additional steps of rinsing the growth media components from the CLC within the cartridge, removing the CLC, together with the substrate support, from the cartridge in preparation for cutting the CLC into sections or units of appropriate sizes for individual product packaging.

Yet another preferred embodiment of this invention includes further processing of the CLC, comprising the additional steps of rinsing the growth media components from the CLC within the cartridge, equilibrating the CLC with cryoprotectant solutions within the cartridge, removing the CLC, together with the substrate support, from the cartridge, in preparation for cutting the CLC into sections or units of appropriate sizes and individually packaging for cryopreservation.

Yet another preferred embodiment of this invention includes further processing of the CLC comprising the additional steps of rinsing the growth media components from the CLC within the cartridge, removing the CLC, together with the substrate support, from the cartridge in preparation for cutting the CLC into sections or units of appropriate sizes, individually packaging and equilibrating the CLC units with cryoprotectant solutions for cyropreservation.

### BRIEF DESCRIPTION OF THE FIGURES

The embodiments and advantages of the present invention will become readily apparent from the detailed description of the invention with reference to the following figures:

FIG. 1 is a flow scheme of the process for making the Composite Living Construct (CLC) of this invention.

FIG. 2 is a cross-sectional view of a preferred embodiment of the bioreactor of this invention.

FIG. 3 is an exploded perspective view of the collagen substrate and its substrate support of the bioreactor of this invention given in FIG. 2.

FIG. 4 is an exploded perspective view of the preferred embodiment of the bioreactor of this invention given in FIG. 2.

FIG. 5 is a cross-sectional view of another preferred embodiment of the bioreactor of this invention.

FIG. 6 is an exploded perspective view of the embodiment of the bioreactor of this invention given in FIG. 5.

FIG. 7 is a flow scheme of the method of using the bioreactor of this invention to make a CLC and for further processing of the CLC.

### DETAILED DESCRIPTION OF THE INVENTION

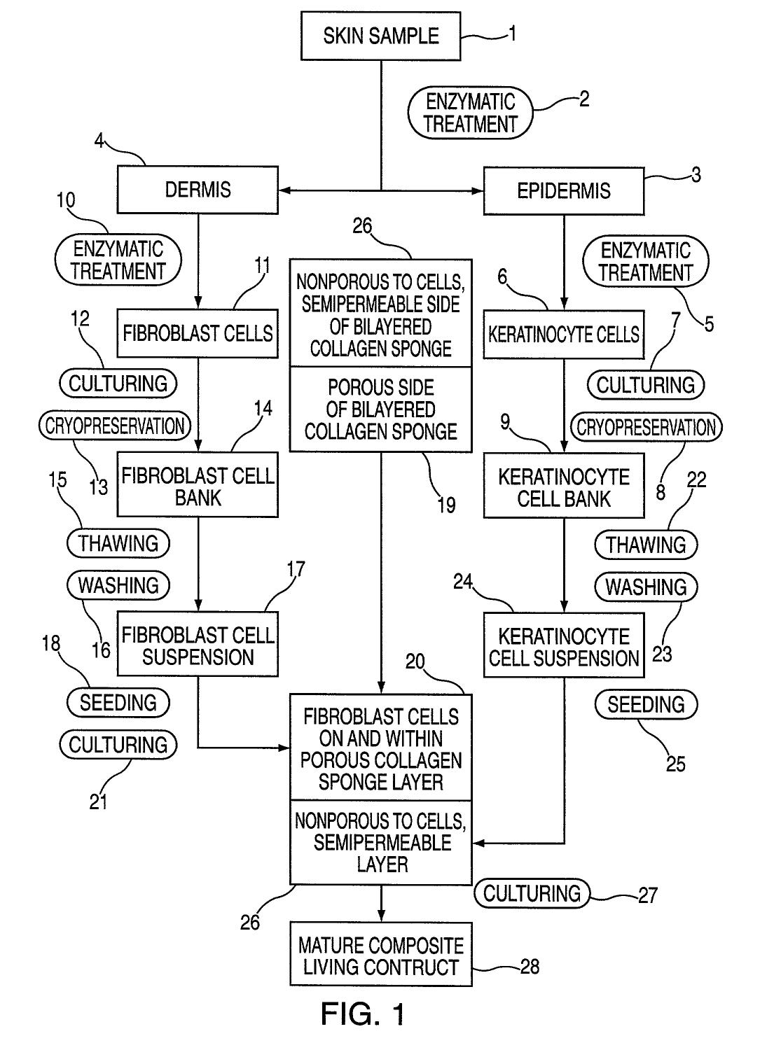

The Composite Living Construct (CLC) 28 of this invention is herein briefly described with reference to FIG. 1, a flow scheme of the process for making the construct. The process has been described in detail in U.S. Pat. Nos. 5,282,859, Re 35,399 and 6,039,859, to Eisenberg, which are entirely incorporated herein by reference. The process for making CLC 28 comprises: treating a skin sample 1 enzymatically 2 to separate the epidermis 3 from dermis 4; treating epidermis 3 enzymatically 5, preferably with trypsin, to release the keratinocyte cells 6; culturing 7 keratinocyte cells 6 to create a cryopreserved 8 keratinocyte cell bank 9; in parallel or sequentially treating dermis 4 enzymatically 10, preferably with collagenase, to release the fibroblast cells 11; culturing 12 fibroblast cells 11 to create a cryopreserved 13 fibroblast cell bank 14; thawing cells 15 from fibroblast cell bank 14; washing 16 cryoprotectants from the cells and preparing a fibroblast cell suspension 17; seeding 18, i.e., inoculating the porous side of a preformed construct 31 comprised of crosslinked collagen sponge 19 with a fibroblast cell suspension 17 to give a fibroblast seeded collagen sponge 20; culturing 21, i.e., incubating, the fibroblast seeded collagen sponge 20 to allow growth of fibroblast cells 11 throughout porous collagen sponge layer 19; and thawing cells 22 from keratinocyte cell bank 9; washing 23 cryoprotectants from the cells and preparing a fibroblast cell suspension 24; seeding 25 the nonporous to cells, semipermeable collagen layer 26 with a keratinocyte cell suspension 24 and then further culturing 27; the composite to yield CLC 28. All the foregoing processes and those following are done under aseptic conditions.

Fibroblasts and keratinocytes of the CLC may be either autologous or allogeneic, the latter enabling the production and storage of inventories of CLCs and thereby eliminating delays in providing CLCs to treat wounds. Media in this invention are used for operations such as equilibrating with the collagen substrate, culturing the fibroblast and keratinocyte cells, seeding the fibroblast and keratinocyte cells, respectively, onto the collagen sponge and the nonporous to cells, semipermeable layers of the collagen substrate, and for further culturing fibroblast and keratinocyte cells within and on the CLC, respectively. Such media comprise Dulbecco's Modified Eagle's Medium (DMEM) and variations thereof, including additional growth factors and other nutrient supplements. The media formulation encourages the growth of cell populations to achieve acceptable values for the following parameters: “cell number”, the total number of cells in CLC; “cell viability”, the percent of the total number of cells that are viable; and “metabolic activity”, a measure of the overall vigor and physiologic state of the viable cells. The preferred growth medium for feeding and incubating, thereby culturing the CLC of this invention, is supplemented DMEM which contains, in addition to DMEM, components such as, but not limited to, fetal bovine serum, recombinant human epidermal growth factor, hydrocortisone, glutamine, cholera toxin, nonessential amino acids (NEAA), NaOH, HEPES (a buffer) and glucose. Spent media are replaced with fresh growth media during culturing. Once the CLC is matured, it may be removed for cutting and packaging or prepared for cryopreservation.

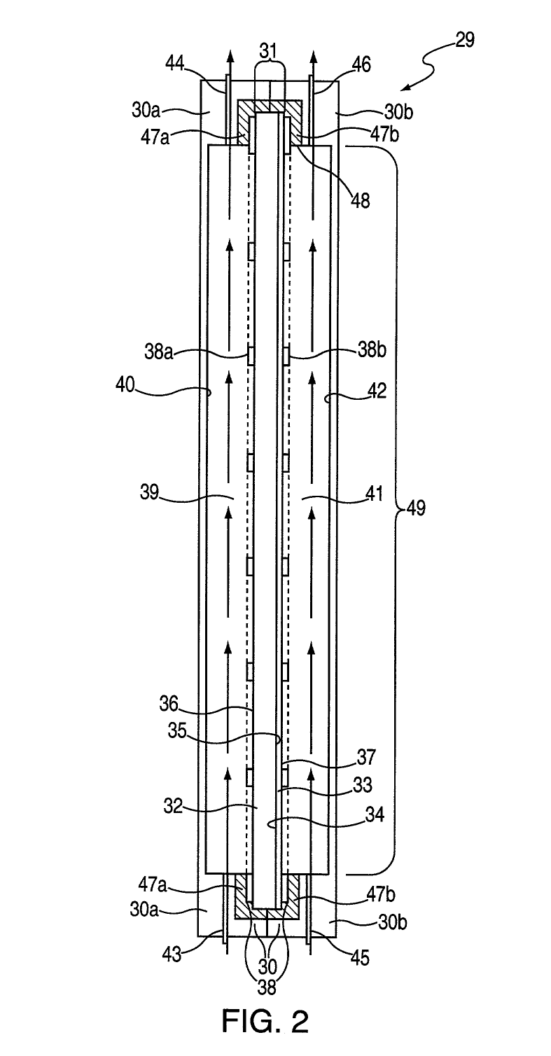

Such CLCs may be made in a first preferred embodiment of the bioreactor of this invention. FIG. 2 is cross-sectional view of such a bioreactor cartridge 29 comprising: at least one cartridge casing 30 having disposed therein a collagen substrate 31 comprised of a porous collagen sponge layer 32 and a nonporous to cells, semipermeable collagen layer 33, each layer having respectively an inner surface 34, 35 in contact with an inner surface of the other layer and an outer surface 36, 37; a substrate support 38 that retains collagen substrate 31 within cartridge casing 30; a first compartment 39 defined between outer surface 36 of collagen sponge layer 32 and the inner surface of the first side of the cartridge casing 40 and a second compartment 41 defined between the outer surface 37 of nonporous to cells, semipermeable collagen layer 33 and the inner surface of the second side of the cartridge casing 42; and a first compartment inlet means 43 and outlet means 44 for transferring a first medium and a first cell type, to first compartment 39 and a second compartment inlet means 45 and outlet means 46 for transferring a second medium and a second cell type, to second compartment 41, inlet and outlet means 43, 44, 45, 46 being situated so that the media are transferred essentially parallel to outer surfaces 36, 37 of layers 32, 33 of collagen substrate 31. In addition to inlet and outlet means 43, 44, 45, 46 for compartments 39, 41, bioreactor 29 may further comprise means for evenly distributing media across the outer surfaces of the layers of the collagen substrate, such as multiple fluid inlet and outlet ports, ribs, channels and wells. It should be noted that every element of bioreactor 29 has a similar opposing component. For example, substrate support 38 is comprised of substrate support grid 38a and a substrate support grid 38b.

With continued reference to FIG. 2, note that bioreactor 29 may comprise numerous collagen substrates 31 sequentially housed, supported by a frame 47 which appropriately mounts the substrate 31 while maintaining the integrity of the first compartment 40 and second compartment 39. That is, a large collagen substrate 31 can be broken down into numerous smaller self-contained units as more fully described in connection with FIG. 8.

With continued reference to FIG. 2, note that bioreactor 29 comprises the following optional additional elements, comprised of opposing components located sequentially and respectively outward from substrate support grid 38a and from substrate support grid 38b; the additional elements having such purposes as support, structural robustness, ease of assembly and disassembly, viewing to the bioreactor contents, allow gas exchange and maintenance of aseptic conditions during use:

A frame 47a and a frame 47b that respectively overlap and support opposing substrate support grids 38a, 38b, each frame being comprised of a border 48 and an opening 49 therewithin. Substrate support grid 38a and frame 47a may optionally be a unitized, combined structure. Similarly, substrate support grid 38b and frame 47b may optionally be a unitized, combined structure. Further optionally, opposing frames 47a, 47b may be combined as a unitized frame, optionally having an inwardly facing channel, that engages both opposing substrate support grids 38a, 38b.

A cartridge casing 30a and a cartridge casing 30b that respectively overlap and support opposing frames 47a, 47b.

Inlet means 43 and outlet means 44 may be situated, so as to provide aseptic access for the first medium and first cell type to first compartment 39, in and through any or combinations of frame 47a, cartridge casing 30a. Similarly, inlet means 45 and outlet means 46 may be situated, so as to provide aseptic access for the second medium and second cell type to second compartment 41, in and through any or combinations of frame 47b, cartridge casing 30b. Multiple inlet and/or multiple outlet means may be provided for access to each compartment. The first and second media are selected from the group consisting of, but not limited to, cell growth media, cell conditioned media, maintenance media, washing media, rinsing media, cell growth factors, enzymes, extracellular matrix components, gases, cryoprotectant solutions and combinations thereof.

It should be noted that some or all components of bioreactor 29, with reference to their having, for example, a rectangular peripheral configuration, may be preassembled for sterilization and/or combined as a unit at least on one side or as many as three sides, thereby permitting insertion, for example, of substrate support 38 and collagen substrate 31 from an open side, which is thereafter sealed closed. Pre-sterilization assembly or aseptic assembly after sterilization may be done by mechanical means such as, but not limited to, clamps, clips, bolts and screws or by adhesive means and may comprise hinges. The peripheral configuration of the cartridge components may be other than rectangular, e.g., square, circular, oval, polygonal and the like.

Bioreactor cartridge 29 and collagen substrate 31 therein are sterilizable by processes such as, but not limited to, Co⁶⁰ radiation, ultraviolet light radiation, ethylene oxide exposure and electron beam radiation. All elements of bioreactor 29 are assembled and sealed to maintain aseptic conditions, either by utilizing the inherent sealing characteristics of their materials of construction such as softness, and surface smoothness, or by the use of gaskets, adhesives and the like. All components of bioreactor cartridge 29 are made of biocompatible, nontoxic plastics, metals and combinations thereof that withstand sterilization processes and the solutions with which they will be in contact during use.

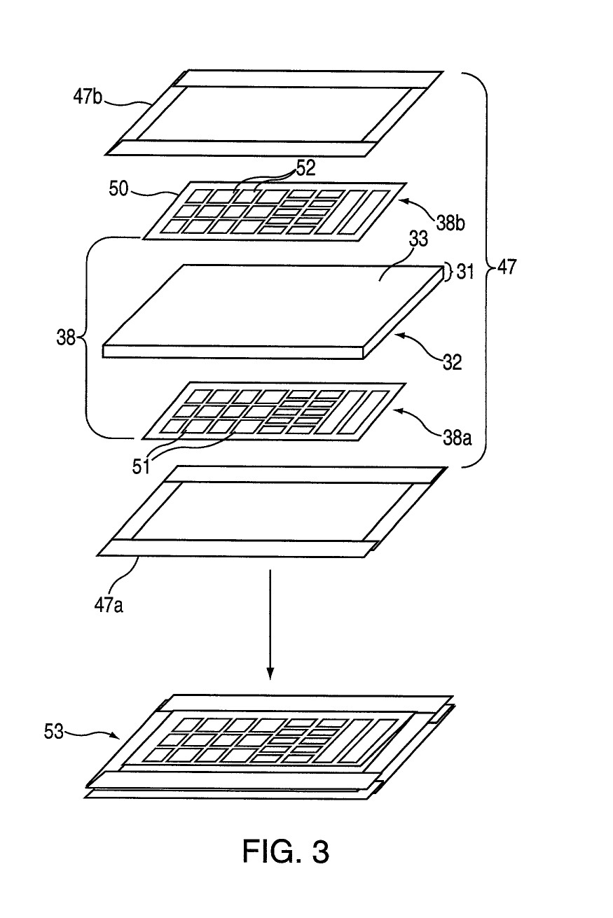

FIG. 3 is an exploded perspective view of the collagen substrate 31, frame 47 and substrate support grid 38 of bioreactor 29 of this invention described in FIG. 2 wherein the common elements of bioreactor 29 have the same numerical designations as are given in FIG. 2. Shown are collagen substrate 31 comprised of porous collagen sponge layer 32 and nonporous to cells, semipermeable collagen layer 33, wherein substrate support grid 38 is respectively comprised of opposing substrate support grids 38a, 38b and frame 47 is respectively comprised of opposing frames 47a, 47b. Substrate support grids 38a, 38b, are preferably each comprised of a component 50 that is respectively adjacent outer surfaces 36, 37 of collagen sponge layer 32 and nonporous to cells, semipermeable collagen layer 33, grid 50 being comprised of solid portions 51 and openings 52 therebetween. It is preferred that openings 51 be configured so as to facilitate dividing, such as by cutting, the Composite Living Construct (CLC) into sections or units, the sections or units preferably being of sizes and configurations that are convenient for use such as for wound dressings. Frames 47a, 47b, are preferably each comprised of a border and an opening therewithin, respectively overlapping and supporting the opposing substrate support grids. Assembly 53 illustrates the frame 47, support grid 38 and substrate 31 assembled as a unit of multiple components to be inserted into the cartridge casing 30 of bioreactor 29.

Collagen sponge layer 32 is preferably comprised of crosslinked collagen. Nonporous to cells, semipermeable collagen layer 33 may be comprised of crosslinked or noncrosslinked collagen, the collagen being selected from the group consisting of, but not limited to, atelocollagen, insoluble collagen and combinations thereof. The nonporous to cells, semipermeable collagen layer is permeable to gases and soluble molecular components and impermeable to biological cells, such as fibroblasts and keratinocytes. The collagen sponge may be of any suitable variety. The collagen for the nonporous to cells, semipermeable collagen layer may be selected from, but not limited to, Type 1 collagen, Type 3 collagen or combinations thereof; an example of such suitable collagen being that from bovine hide obtainable from CTI (Cohesion Technology Incorporated).

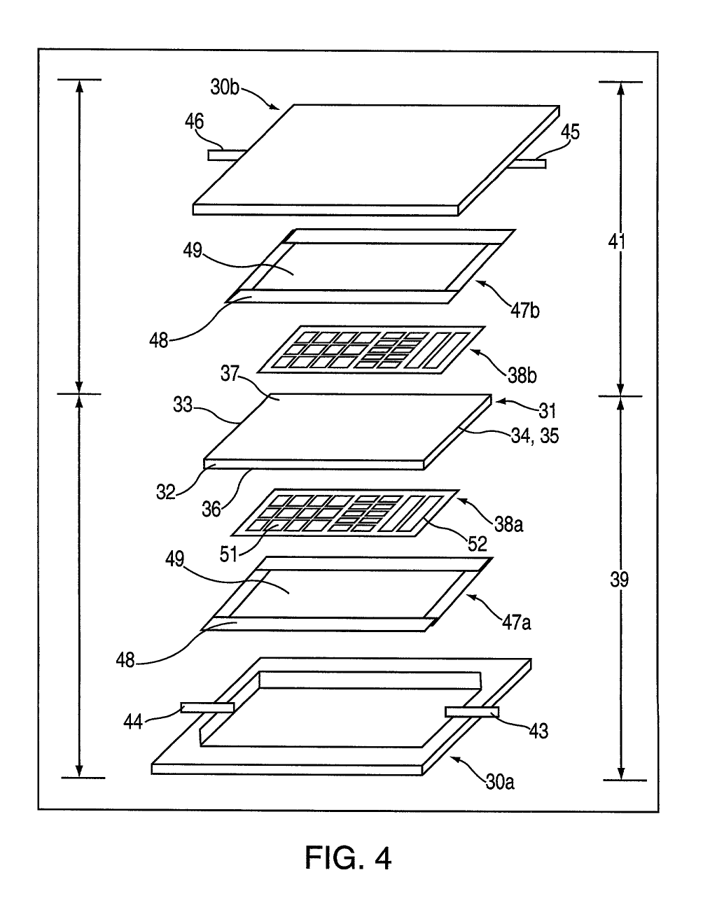

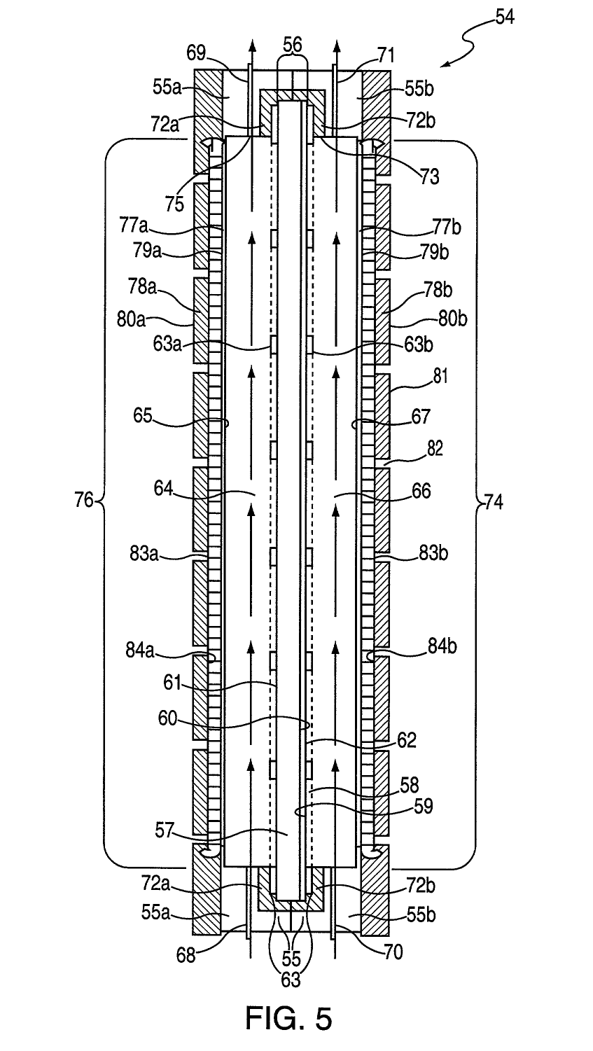

FIG. 4 is an exploded (not to scale) perspective view of the preferred embodiment of bioreactor 29 of this invention given in FIG. 2, wherein the elements of bioreactor 29 have the same numerical designations as are given in FIG. 2.

FIG. 5 is cross-sectional view of another preferred embodiment of a bioreactor cartridge 54 of this invention comprising: at least one cartridge casing 55 having disposed therein a collagen substrate 56 comprised of a porous collagen sponge layer 57 and a nonporous to cells, semipermeable collagen layer 58, each layer having respectively an inner surface 59,60 in contact with an inner surface of the other layer and an outer surface 61,62; a substrate support 63, comprised of opposing substrate support grids 63a, 63b, that retain collagen substrate 56 within bioreactor cartridge 54; a first compartment 64 defined between outer surface 61 of porous collagen sponge layer 57 and the inner surface of cartridge casing 65 and a second compartment 66 defined between outer surface 62 of nonporous to cells, semipermeable collagen layer 58 and the inner surface of cartridge casing 67; and a first compartment inlet means 68 and outlet means 69 for transferring a first medium and a first cell type to first compartment 64 and a second compartment inlet means 70 and outlet means 71 for transferring a second medium and a second cell type to second compartment 66, inlet and outlet means 68, 69, 70, 71 being situated so that the media are transferred essentially parallel to outer surfaces 61, 62 of layers 57, 58 of collagen substrate 56.

Cartridge 54 comprises the following additional elements, comprised of opposing components located sequentially and respectively outward from substrate support 63a and substrate support 63b:

A frame 72a, and a frame 72b that respectively overlap and support opposing substrate support grids 63a, 63b, each frame being comprised of a border 73 and an opening 74 therewithin.

A cartridge casing 55a and cartridge casing 55b that respectively overlap and support opposing frames 72a, 72b, each casing being comprised of a casing border 75 and an opening 76 therewithin.

The openings within cartridge casings 55a, 55b are sealed with opposing gas permeable, liquid impermeable membranes 77a, 77b so as to maintain aseptic conditions during use of bioreactor 54. Opposing membranes 77a, 77b permit the aseptic transfer of gases such as, but not limited to, air, oxygen and carbon dioxide into and out of the bioreactor. Such membranes may conveniently be formed from gas permeable materials such as, but not limited to, silicone polymers, polyurethanes and combinations thereof.

When, as shown in FIG. 5, a portion of the cartridge casing is comprised of gas permeable, liquid impermeable membranes 77a, 77b, a window 78a and a window 78b may be employed to provide structural support to each membrane surface 79a, 79b of opposing cartridge casings 55a, 55b by fitting over the membrane surface 79a, 79b, each window having an outside surface 80a, 80b and comprised of solid portions 81 and openings 82 therebetween, the openings 82 designed to allow gas flow between the windows 77a, 77b and the gas permeable membranes 77a, 77b, while maintaining direct and unobstructed view respectively of first and second compartments 64, 66 and their contents.

When, as shown in FIG. 5, a portion of the cartridge casing is comprised of gas permeable, liquid impermeable membranes 77a, 77b, mesh supports 83a, 83b may be employed between the outer surface of opposing membranes 79a, 79b and the inner surface of opposing windows 84a, 84b to maintain adequate air flow between windows 78a, 78b and membranes 77a, 77b. Membranes 77a, 77b and mesh supports 83a, 83b are preferably of such material and porosity and/or transparency so as not to obstruct easy viewing of the contents of bioreactor 54.

If membranes 77a, 77b and mesh supports 83a, 83b, have sufficient self supporting stiffness and structural integrity, windows 78a, 78b may optionally be eliminated, care being taken to seal each membrane, at least around its periphery, to the cartridge casing to maintain aseptic conditions during use of bioreactor 54.

A multiple cartridge bioreactor system may have the following fluid pathway alternatives: for an independent cartridge arrangement, the fluid flow path to the first compartment of each cartridge is separate from that of the first compartment of the other cartridges and the fluid flow path to the second compartment of each cartridge is separate from that of the second compartment of the other cartridges; for a series cartridge arrangement, the fluid flow path to the first compartment of each cartridge is connected in series and the fluid flow path to the second compartment of each cartridge is connected in series; for a parallel cartridge arrangement, the fluid flow path to the first compartment of each cartridge is connected in parallel and the fluid flow path to the second compartment of each cartridge is connected in parallel; for a combination parallel and series arrangement, groups of cartridges are connected in a parallel arrangement as described above, while the fluid flow path from the first compartment of each of these grouped cartridges is connected in series with the fluid flow path to the respective first compartment of the cartridges in the remaining parallel groups and the fluid flow path from the second compartment of each of these grouped cartridges is connected in series with the fluid flow path to the respective second compartment of each of the cartridges in the remaining parallel groups. The fluid pathways of the first and second compartments of each cartridge optionally may be connected with each other to form a unitary fluid pathway. Optionally, the multiple bioreactor system may have any of the above mentioned fluid flow path arrangements with the exception that the fluid pathway connects only the first compartments of each cartridge, or the fluid pathway connects only the second compartments of each cartridge.

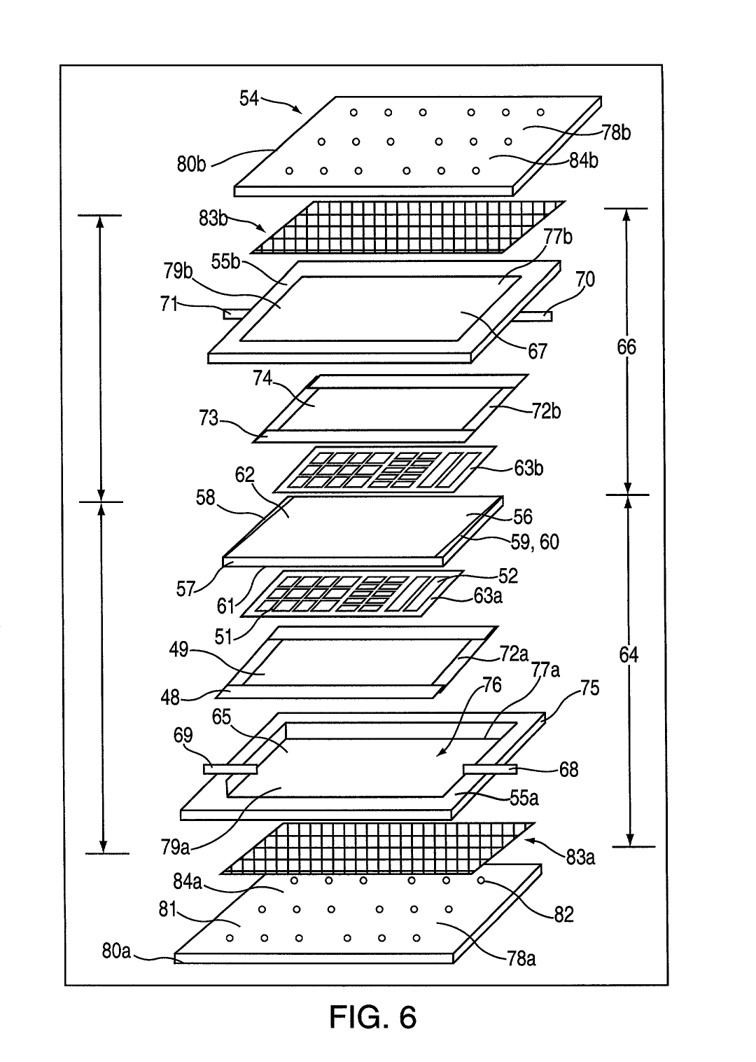

FIG. 6 is an exploded (not to scale) perspective view of the preferred embodiment of bioreactor 54 of this invention given in FIG. 5, wherein the elements of bioreactor 54 have the same numerical designations as are given in FIG. 5.

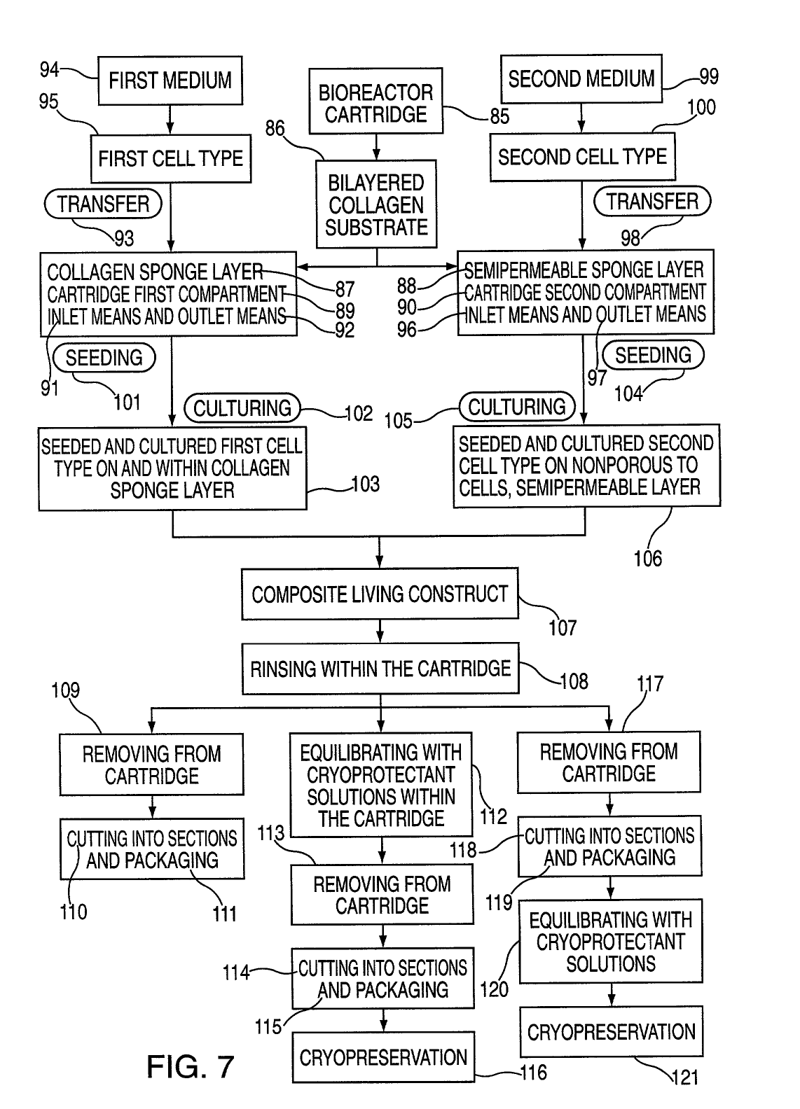

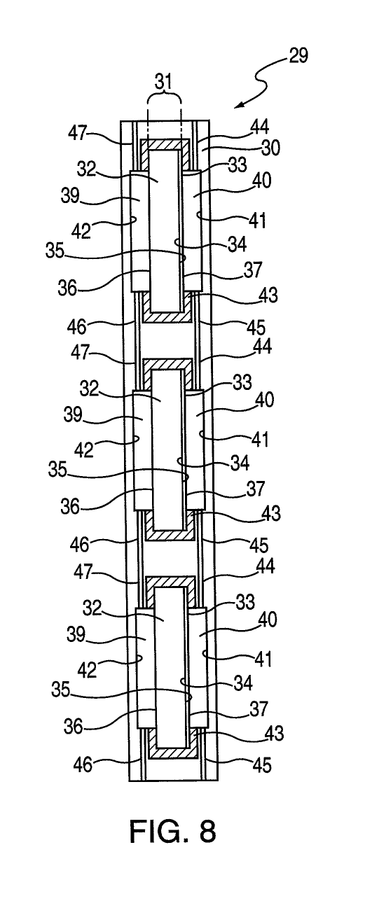

Yet another preferred embodiment of this invention, described with reference to FIG. 7, is a flow scheme of a method for making a CLC 107 comprised of at least a first layer comprising a cultured first cell type 95 and at least a second layer comprising a cultured second cell type 100, the method comprising the steps of:

1) providing a bioreactor system comprising: at least one bioreactor cartridge 85 having disposed therein a collagen substrate 86 comprised of a collagen sponge layer 87 and a nonporous to cells, semipermeable collagen layer 88, each layer respectively having an inner surface in contact with an inner surface of the other layer and an outer surface; a substrate support that retains the collagen substrate 86 within cartridge 85; a first compartment 89 defined between the outer surface of collagen sponge layer 87 and the inner surface of cartridge casing and a second compartment 90 defined between the outer surface of nonporous to cells, semipermeable collagen layer 88 and the inner surface of cartridge casing; and a first compartment inlet means 91 and outlet means 92 for transferring 93 a first medium 94 and a first cell type 95 to the first compartment 89 and a second compartment inlet means 96 and outlet means 97 for transferring 98 a second medium 99 and a second cell type 100 to second compartment 90, the inlet and outlet means being situated so that media 94, 99 and cell types 95, 100 are respectively transferred essentially parallel to the outer surfaces of layers 87, 88 of collagen substrate 86;

2) transferring 93 first medium 94 and first cell type 95 to first compartment 89;

3) effecting a seeding 101 and culturing 102 of first cell type 95 on collagen sponge layer 103;

4) transferring 98 second medium 99 and second cell type 100 to second compartment 90; and

5) effecting a seeding 104 and culturing 105 of second cell type 100 on the nonporous to cells, semipermeable collagen layer 106;

First and second media 94, 99 are selected from the group consisting of, but not limited to, cell growth media, cell conditioned media, maintenance media, washing media, rinsing media, cell growth factors, enzymes, extracellular matrix components, gases, cryoprotectant solutions and combinations thereof; and may be identical to or different from one another. Gases may be supplied to the first and second media prior to transferring them respectively to first and second compartments 89, 90. Gases may alternatively and/or additionally be transferred to media 94, 99, via a gas permeable, liquid impermeable membrane that comprises a portion of cartridge 85, when media 89, 90 are in their respective compartments 89, 90 and while seeding and culturing 101, 102, 104, 105 of cell types 95, 100 are being effected on respective collagen layers 87, 88. Gases are those commonly encountered in cell culture being selected from the group consisting of, but not limited to, oxygen, nitrogen, carbon dioxide and combinations thereof. It is preferred that method in FIG. 7 further comprise maintaining within bioreactor 85 a pressure equal to or greater than one atmosphere.

It is preferred that the steps of transferring 93 first medium 94 and first cell type 95 to first compartment 89 and transferring 98 second medium 99 and second cell type 100 to second compartment 90 are each performed with the major surface of the collagen substrate being between about 45° and 90° from the horizontal, the inlet and outlet means being situated so as to minimize entrapment of gases within the cartridge. It is also preferred the steps of effecting a seeding and culturing 101, 102 of first cell type 95 on collagen sponge layer 87 and effecting a seeding and culturing 104, 105 of second cell type 100 on the nonporous to cells, semipermeable collagen layer 106 are each done with the major surface of the collagen substrate being between about 45° and 0° from the horizontal. It should be noted that steps 4) 98, and 5) 104 and 105 may alternatively and conveniently precede steps 2) 93 and 3) 101 and 102.

Still further embodiments of this invention are described in FIG. 7, a flow scheme of options for further processing a CLC 107 made by the method of this invention given in FIG. 7.

One further processing option given in FIG. 7, comprises the steps of:

a) rinsing 108 the growth media components from the CLC within the cartridge

b) removing 109 the CLC along with the substrate support from the cartridge

c) cutting 110 the CLC 107 into sections or units and then packaging 111 the individual sections or units of CLC 107;

Yet another further processing option given in FIG. 7, comprises the steps of:

a) rinsing 108 the growth media components from the CLC within the cartridge;

b) equilibrating 112 CLC 107 with cryoprotectant solutions within the cartridge and either directly cryopreserving within the cartridge thus providing packaging for the product or alternatively proceeding to step c);

c) removing 113 the CLC along with the substrate support from the cartridge;

d) cutting 114 equilibrated CLC 107 into sections or units and packaging 115 the sections or units individually;

e) cryopreserving 116 individually packaged CLC sections or units and storing.

Yet another further processing option given in FIG. 7, comprises the steps of:

a) rinsing 108 the growth media components from the CLC within the cartridge;

b) removing 117 the CLC along with the substrate support from the cartridge;

c) cutting 118 CLC 107 into sections or units and packaging 119 the sections or units individually;

d) equilibrating 120 the individually packaged CLC units with cryoprotectant solutions;

e) cryopreserving 121 individually packaged CLC sections or units and storing.

As further embodiments of the bioreactor of the present invention, CLC's may be provided as discrete small self-contained cartridges within an overall composite frame. Such CLC's are shown in a yet further preferred embodiment of the bioreactor of this invention in FIG. 8.

FIG. 8 is a cross-sectional view of such a bioreactor cartridge 29 comprising: at least one cartridge casing 30 having disposed therein at least one collagen substrate 31 comprised of a porous collagen sponge layer 32 and a nonporous to cells, semipermeable collagen layer 33 the substrate being held by frame 43, each layer having respectively an inner surface 34, 35 in contact with an inner surface of the other layer and an outer surface 36, 37; a first compartment 39 defined between outer surface 36 of collagen sponge layer 32 and the inner surface of the first side of the cartridge casing 42 and a second compartment 40 defined between the outer surface 37 of the nonporous to cells, semipermeable collagen layer 33 and the inner surface of the second side of the cartridge casing 41; and a first compartment inlet means 44 and outlet means 45 for transferring a first medium and a first cell type, to first compartment 40 and a second compartment inlet means 47 and outlet means 46 for transferring a second medium and a second cell type, to second compartment 39, inlet and outlet means 44, 45, 46, 47 being situated so that the media are transferred essentially parallel to outer surfaces 36, 37 of layers 32, 33 of collagen substrate 31.

While inlet means 47 and 44 are shown in FIG. 8 as being contiguous with outlet means 46 and 45, respectively, it is also an option of the invention to dedicate each inlet means and outlet means to each cartridge 39 separately. This would provide multiple sets of inlet ports and outlet ports, one for each separate cartridge.

Although the foregoing has been described with respect to the CLC being a porous collagen sponge layer and a nonporous to cells, semi permeable collagen layer, it will be apparent to those skilled in the art that the bioreactor will be operative with respect to CLCs constructed of different materials which satisfy the criteria set forth for the collagen/collagen construct illustrated herein, such as collagen/glycosaminoglycans constructs.

We claim:

1. A bioreactor comprising
a) at least one cartridge having disposed therein:
i. a collagen substrate comprised of a collagen sponge layer and a nonporous to cells, semipermeable collagen layer, each layer having an inner surface in contact with an inner surface of the other layer and an outer surface;
ii. a substrate support that retains the collagen substrate within the cartridge;
iii. a first compartment defined between the outer surface of the collagen sponge layer and the inner surface of one side of the cartridge and a second compartment defined between the outer surface of the nonporous to cells, semipermeable collagen layer and the inner surface of a second side of the cartridge; and
iv. a first compartment inlet means and outlet means for transferring a first medium and a first cell type to the first compartment and a second compartment inlet means and outlet means for transferring a second medium and a second cell type to the second compartment, the inlet and outlet means being situated so that the media and cell types are transferred essentially parallel to the layers of the collagen substrate.

2. The bioreactor of claim 1 wherein the collagen sponge and nonporous to cells, semipermeable collagen layers are comprised of crosslinked collagen.

3. The bioreactor of claim 1 wherein the collagen sponge layer is comprised of crosslinked collagen and the nonporous to cells, semipermeable collagen layer is comprised of noncrosslinked collagen.

4. The bioreactor of claim 1 wherein the nonporous to cells, semipermeable collagen layer is selected from the group consisting of, but not limited to, atelocollagen, insoluble collagen and combinations thereof.

5. The bioreactor of claim 1 wherein the nonporous to cells, semipermeable collagen layer is impermeable at least to fibroblasts and keratinocytes.

6. The bioreactor of claim 1 wherein at least a portion of the cartridge surface is comprised of a gas permeable, liquid impermeable membrane.

7. The bioreactor of claim 6 wherein the membrane is selected from, but not limited to, the group consisting of silicone polymers, polyurethanes and combinations thereof.

8. The bioreactor of claim 6 wherein the membrane allows exchange of gases selected from, but not limited to, the group consisting of air, oxygen, nitrogen, carbon dioxide and combinations thereof.

9. The bioreactor of claim 1 wherein the substrate support is a frame that secures the substrate peripherally within the cartridge.

10. The bioreactor of claim 1 wherein the substrate support is two opposing grids adjacent to the major outer surfaces of the collagen substrate, each grid having a surface comprised of solid portions and openings therebetween and being secured peripherally within a support frame.

11. The bioreactor of claim 10 wherein the openings of the support grid are configured to facilitate cutting of the Composite Living Constructs (CLCs) along the periphery of the openings.

12. The bioreactor of claim 1 comprising the cartridge, the collagen substrate and the substrate support having been sterilized separately and then aseptically assembled.

13. The bioreactor of claim 1 further comprising the cartridge containing the collagen substrate and the substrate support having been assembled and then subjected to a sterilization process.

14. The bioreactor of claim 1 wherein the sterilization process is selected from, but not limited to the group consisting of Co⁶⁰ irradiation, ultraviolet light irradiation, ethylene oxide sterilization and electron beam irradiation.

15. The bioreactor of claim 1 wherein the first and second media are selected from, but not limited to, the group consisting of cell growth media, cell conditioned media, maintenance media, washing media, rinsing media, cell growth factors, enzymes, extracellular matrix components, gases, cryoprotectant solutions and combinations thereof.

16. The bioreactor of claim 1 wherein the first cell type comprises fibroblasts and the second cell type comprises keratinocytes.

17. The bioreactor of claim 1 which further comprises means for evenly distributing fluid across the outer surfaces of the layers of the collagen substrate, such means being selected from, but not limited to, the group consisting of fluid manifolds, ribs, channels, wells, the spatial orientations of the fluid manifold, ribs, channels and wells and combinations thereof.

18. The bioreactor of claim 1, which further comprises multiple cartridges.

19. The bioreactor of claim 18 wherein the cartridges are connected in an independent cartridge arrangement, where the fluid flow path to the first compartment of each cartridge is separate from that of the first compartment of the other cartridges and the fluid flow path to the second compartment of each cartridge is separate from that of the second compartment of the other cartridges.

20. The bioreactor of claim 18 wherein the cartridges are connected in a series cartridge arrangement, where the fluid flow path to the first compartment of each cartridge is connected in series and the fluid flow path to the second compartment of each cartridge is connected in series.

21. The bioreactor of claim 18 wherein the cartridges are connected in a parallel cartridge arrangement, where the fluid flow path to the first compartment of each cartridge is connected in parallel and the fluid flow path to the second compartment of each cartridge is connected in parallel.

22. The bioreactor of claim 18 wherein the cartridges are connected in a combination series and parallel cartridge arrangement, where groups of cartridges are connected in a parallel arrangement as described above, while the fluid flow path from the first compartment of each of these grouped cartridges is connected in series with the fluid flow path to the respective first compartment of the cartridges in the remaining parallel groups and the fluid flow path from the second compartment of each of these grouped cartridges is connected in series with the fluid flow path to the respective second compartment of each of the cartridges in the remaining parallel groups.

23. The bioreactor of claim 18 wherein the fluid pathways of the first and second compartments of each cartridge optionally may be connected with each other to form a unitary fluid pathway.

24. Optionally, the bioreactor of claim 18 may have any of the above mentioned fluid flow path arrangements with the exception that the fluid pathway connects only the first compartments of each cartridge, or the fluid pathway connects only the second compartments of each cartridge.

25. A method of making a Composite Living Construct (CLC) comprised of at least a first layer comprising a cultured first cell type and at least a second layer comprising a cultured second cell type, the method comprising the steps of:
a) providing a bioreactor comprising
i. at least one cartridge having disposed therein a collagen substrate comprised of a collagen sponge layer and a nonporous to cells, semipermeable collagen layer, each layer having an inner surface in contact at a common plane with an inner surface of the other layer and an outer surface;
ii. a substrate support that retains the collagen substrate within the cartridge;
iii. a first compartment defined between the outer surface of the collagen sponge layer and the inner surface of one side of the cartridge and a second compartment defined between the outer surface of the nonporous to cells, semipermeable collagen layer and the inner surface of a second side of the cartridge; and
iv. a first compartment inlet means and outlet means for transferring a first medium and a first cell type to the first compartment and a second compartment inlet means and outlet means for transferring a second medium and a second cell type to the second compartment, the inlet and outlet means being situated so that the media and cell types are transferred essentially parallel to the layers of the collagen substrate;
b) transferring the first medium and the first cell type to the first compartment;
c) effecting a seeding and culturing of the first cell type on the collagen sponge layer;
d) transferring the second medium and the second cell type to the second compartment; and
e) effecting a seeding and culturing of the second cell type on the nonporous to cells, semipermeable collagen layer.

26. The method of claim 25 wherein the collagen sponge and nonporous to cells, semipermeable collagen layers are comprised of crosslinked collagen.

27. The method of claim 25 wherein the collagen sponge layer is comprised of crosslinked collagen and the nonporous to cells, semipermeable collagen layer is comprised of noncrosslinked collagen.

28. The method of claim 25 wherein the nonporous to cells, semipermeable collagen layer is selected from, but not limited to, the group consisting of atelocollagen, insoluble collagen and combinations thereof.

29. The method of claim 25 wherein the nonporous to cells, semipermeable collagen layer is impermeable at least to fibroblasts and keratinocytes.

30. The method of claim 25 wherein the steps d) and e) precede steps b) and c).

31. The method of claim 25 wherein the steps of transferring the first medium and the first cell type to the first compartment and transferring the second medium and the second cell type to the second compartment are each performed with the major surface of the collagen substrate being between about 45° and 90° from the horizontal, the inlet and outlet means being situated so as to minimize entrapment of gases within the cartridge.

32. The method of claim 25 wherein the steps of effecting a seeding and culturing of the first cell type on the collagen sponge layer and effecting a seeding and culturing of the second cell type on the nonporous to cells, semipermeable collagen layer are each done with the major surface of the collagen substrate being between about 45° and 0° from the horizontal.

33. The method of claim 25 where in step c) the culturing of the first cell type is additionally effected within the collagen sponge layer.

34. The method of claim 25 wherein the substrate support is a frame that secures the substrate peripherally within the cartridge.

35. The method of claim 25 wherein the substrate support is two opposing grids adjacent to the major surfaces of the collagen substrate, each grid having a surface comprised of solid portions and openings therebetween and secured peripherally within a support frame.

36. The method of claim 35 wherein the openings of the grid are configured to facilitate cutting of the CLC along the periphery of the openings.

37. The method of claim 25 comprising the cartridge, the collagen substrate and the substrate support having been sterilized separately and then aseptically assembled.

38. The method of claim 25 further comprising the cartridge containing the collagen substrate and the substrate support having been assembled and then subjected to a sterilization process.

39. The method of claim 37 wherein the sterilization process is selected from, but not limited to, the group consisting of Co⁶⁰ irradiation, ultraviolet light irradiation, ethylene oxide sterilization and electron beam irradiation.

40. The method of claim 25 wherein the first and second media are selected from, but not limited to, the group consisting of cell growth media, cell conditioned media, maintenance media, washing media, rinsing media, cell growth factors, enzymes, extracellular matrix components, gases, cryoprotectant solutions and combinations thereof.

41. The method of claim 40 wherein the gases are supplied to the first medium prior to transferring the first medium to the first compartment.

42. The method of claim 40 wherein the gases are supplied to the second medium prior to transferring the second medium to the second compartment.

43. The method of claim 40 wherein the gases are supplied to the first medium while effecting the seeding and culturing of the first cell type on the collagen sponge layer.

44. The method of claim 40 wherein the gases are supplied to the second medium while effecting the seeding and culturing of the second cell type on the nonporous to cells, semipermeable collagen layer.

45. The method of claim 25 wherein the first cell type comprises fibroblasts and the second cell type comprises keratinocytes.

46. The method of claim 25 wherein the bioreactor further comprises at least a portion of the cartridge being a gas permeable, liquid impermeable membrane.

47. The method of claim 46 wherein the membrane is selected from, but not limited to, the group consisting of silicone polymers, polyurethanes and combinations thereof.

48. The method of claim 46 wherein the membrane allows exchange of gases selected from, but not limited to, the group consisting of oxygen, nitrogen, carbon dioxide and combinations thereof.

49. The method of claim 25, which further comprises maintaining within the bioreactor a pressure equal to or greater than one atmosphere.

50. The method of claim 25 which further comprises the step of equilibrating the CLC with a cryoprotectant after having effected both the culturing of the first cell type within the collagen sponge layer and the culturing of the second cell type on the nonporous to cells, semipermeable collagen layer.

51. The method of claim 25 performed under aseptic conditions.

52. The method of claim 33 performed under aseptic conditions.

53. The method of claim 34 performed under aseptic conditions.

54. The method of claim 35 performed under aseptic conditions.

55. The method of claim 36 performed under aseptic conditions.

56. The method of claim 41 performed under aseptic conditions.

57. The method of claim 42 performed under aseptic conditions.

58. The method of claim 43 performed under aseptic conditions.

59. The method of claim 44 performed under aseptic conditions.

60. The method of claim 49 performed under aseptic conditions.

61. The method of claim 50 performed under aseptic conditions.
